(19) United States
(12) Patent Application Publication
(10) Pub. No.: US 2018/0256745 A1
(43) Pub. Date: Sep. 13, 2018

(54) BIOMIMETIC ARTIFICIAL CELLS: ANISOTROPIC SUPPORTED LIPID BILAYERS ON BIODEGRADABLE MICRO AND NANOPARTICLES FOR SPATIALLY DYNAMIC SURFACE BIOMOLECULE PRESENTATION

(71) Applicant: THE JOHNS HOPKINS UNIVERSITY, BALTIMORE, MD (US)

(72) Inventors: RANDALL A. MEYER, BALTIMORE, MD (US); MOHIT MATTHEW, BALTIMORE, MD (US); JOEL C. SUNSHINE, PIKESVILLE, MD (US); RON B. SHMUELI, BALTIMORE, MD (US); JORDAN J. GREEN, NOTTINGHAM, MD (US); KEVIN YAREMA, BALTIMORE, MD (US)

(73) Assignee: THE JOHNS HOPKINS UNIVERSITY, BALTIMORE, MD (US)

(21) Appl. No.: 15/561,879

(22) PCT Filed: Mar. 28, 2016
(86) PCT No.: PCT/US2016/024500

§ 371 Date: Sep. 26, 2017

Related U.S. Application Data
(60) Provisional application No. 62/138,707, filed on Mar. 26, 2015.

(51) Int. Cl. A61K 47/69; A61K 9/127; A61K 9/16; C07K 16/28

(52) U.S. Cl. CPC A61K 47/6911; A61K 47/6937; C07K 16/2818; A61K 9/1647; A61K 9/1274

(57) ABSTRACT

The presently disclosed subject matter provides compositions and methods for using a non-spherical biomimetic artificial cell comprising a three-dimensional microparticle or nanoparticle having an asymmetrical shape and a supported lipid bilayer (SLB). The non-spherical biomimetic artificial cells can be used in cell biomimicry and for active targeting mediated drug delivery.

### CROSS-REFERENCE TO RELATED APPLICATIONS

This application claims the benefit of U.S. Provisional Application No. 62/138,707, filed Mar. 26, 2015, which is incorporated herein by reference in its entirety.

### FEDERALLY SPONSORED RESEARCH OR DEVELOPMENT

This invention was made with government support under R01-EB016721 and R25CA153952 awarded by the National Institutes of Health (NIH). The government has certain rights in the invention.

### BACKGROUND

Lipid polymer hybrid particles have been of great interest to the biomaterials community in recent years. This strategy of particle synthesis combines the biomimetic cellular surface features of a liposome with the structural support and long term stability of a polymeric particle. The technology has proven to be promising for several applications (Hadinoto et al., 2013) including drug delivery (Cheow and Hadinoto, 2011), diagnostic imaging (Willem, 2012), and gene delivery (Xu et al., 2013; Hasan et al., 2011). Generally, these constructs are of core-shell design with the polymer encapsulating various therapeutics in the core, and lipids forming a supported lipid bilayer (SLB) shell. By fusing a preformed liposome to polymeric particle (Wang et al., 2010) or taking advantage of self-assembling lipid bilayers during particle synthesis (Bershteyn et al., 2008), these particles can be fabricated with a variety of different strategies depending on the desired application.

Despite the promise of the lipid polymer hybrid particle for various therapeutic applications, to date almost all particle SLBs have been based on the use of spherical particles to serve as a template for the shape of the SLB. Recent research into particle shape suggests non-spherical particles are superior to spherical particles given their ability to avoid non-specific cellular uptake and their capability to enhance targeted specific cellular uptake (Canelas et al., 2009). In addition, non-spherical particles can be produced from a variety of accessible top-down (Canelas et al., 2009) and bottom-up methods (Champion et al., 2007). As such, non-spherical particles also have found application in fields, such as drug delivery (Karagoz et al., 2014; Chu et al., 2014), gene delivery (Xu et al., 2013; Jiang et al., 2011; Jiang et al., 2013), and immunoengineering (Sunshine et al., 2014).

### SUMMARY

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning. A Laboratory Manual, 3^{rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange 10^{th} ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, World Wide Web URL: http://www.ncbi.nlm.nih.gov/omim/ and in Online Mendelian Inheritance in Animals (OMIA), a database of genes, inherited disorders and traits in animal species (other than human and mouse), at http://omia.angis.org.au/contact.shtml.

In some aspects, the presently disclosed subject matter provides a non-spherical biomimetic artificial cell comprising: (a) a three-dimensional microparticle or nanoparticle having an asymmetrical shape; (b) a supported lipid bilayer (SLB); and (c) at least one biomolecule conjugated to the SLB.

In other aspects, the presently disclosed subject matter provides a non-spherical biomimetic artificial cell comprising: (a) a three-dimensional microparticle or nanoparticle having an asymmetrical shape, wherein the asymmetrical shape has at least one surface having a radius of curvature along at least one axis selected from one of the following ranges: (a) about 1 nm to about 10 nm; (b) about 11 nm to about 100 nm; (c) about 101 nm to about 400 nm; (d) about 401 nm to about 1μm; (e) about 10 μm to about 20 μm; (f) about 20 μm to about 100 μm; and (g) about 101 μm to about 1 mm; (b) a supported lipid bilayer (SLB); and (c) at least one biomolecule conjugated to the SLB.

In yet other aspects, the presently disclosed subject matter provides a non-spherical biomimetic artificial cell comprising: (a) a three-dimensional microparticle or nanoparticle having an asymmetrical shape defined by a dimension (a) along an x-axis, a dimension (b) along a y-axis, and a dimension (c) along a z-axis, wherein at least one of (a), (b), or (c) is not equal to at least one other dimension (a), (b), or (c); (b) a supported lipid bilayer (SLB); and (c) at least one biomolecule conjugated to the SLB.

In some embodiments, the non-spherical biomimetic artificial cell has an ellipsoidal shape. In some embodiments, the three-dimensional microparticle or nanoparticle comprises an ellipsoid selected from the group consisting of: a prolate ellipsoid, wherein the dimension (a) along the x-axis is greater than the dimension (b) along the y-axis, and wherein the dimension (b) along the y-axis is equal to the dimension (c) along the z-axis, such that the prolate ellipsoid can be described by the equation a>b=c; a tri-axial ellipsoid, wherein the dimension (a) along the x-axis is greater than the dimension (b) along the y-axis, and wherein the dimension (b) along the y-axis is greater than the dimension (c) along the z-axis, such that the tri-axial ellipsoid can be described by the equation a>b>c; and an oblate ellipsoid, wherein the dimension (a) along the x-axis is equal to the dimension (b) along the y-axis, and wherein the dimension (b) along the y-axis is greater than the dimension (c) along the z-axis, such that the oblate ellipsoid can be described by the equation a=b>c.

In some embodiments, the three-dimensional microparticle or nanoparticle comprises a material having one or more of the following characteristics: (i) one or more degradable linkages; (ii) a stretchable modulus; and (iii) a glass transition temperature such that the material comprising the three-dimensional microparticle or nanoparticle is a solid at room temperature and/or body temperature. In some embodiments, the degradable linkage is selected from the group consisting of an ester linkage, a disulfide linkage, an amide linkage, an anhydride linkage, and a linkage susceptible to enzymatic degradation.

In some embodiments, the microparticle or nanoparticle comprises a biodegradable polymer or blends of polymers selected from the group consisting of poly(lactic-co-glycolic acid) (PLGA), poly(beta-amino ester) (PBAE), polycaprolactone (PCL), polyglycolic acid (PGA), polylactic acid (PLA), poiy(acrylic acid) (PAA), poly-3-hydroxybutyrate (P3HB), and poly(hydroxybutyrate-co-hydroxyvalerate). In some embodiments, the microparticle or nanoparticle further comprises a biodegradable polymer or blends of polymers blended with a nondegradable polymer. In some embodiments, the microparticle or nanoparticle has an aspect ratio ranging from about 1.1 to about 5.

In some embodiments, at least one biomolecule conjugated to the SLB is found on the surface of the SLB. In some embodiments, at least one biomolecule conjugated to the SLB comprises at least one entity selected from the group consisting of a drug, therapeutic agent, protein, peptide, sugar and polysaccharide. In some embodiments, at least one biomolecule is a protein or a fragment thereof. In some embodiments, the protein or a fragment thereof is a biotin-binding protein or fragment thereof. In some embodiments, the biotin-binding protein or fragment thereof is selected from the group consisting of avidin, streptavidin, neutravidin, and fragment thereof.

In some embodiments, the SLB retains lateral diffusive properties as compared to the SLB of a similar spherical biomimetic artificial cell. In some embodiments, the membrane fluidity is selected from the group consisting of 10e-7 to 10e-8 cm²/s, 10e-8 to 10e-9 cm²/s, 10e-9 to 10e-10 cm²/s, 10e-10 to 10e-11 cm²/s, and 10e-11 to 10e-12 cm²/s.

In some embodiments, the non-spherical biomimetic artificial cell is more resistant to phagocytosis as compared to a similar spherical biomimetic artificial cell. In some embodiments, the non-spherical biomimetic artificial cell is capable of interacting with a T cell receptor.

In some aspects, the presently disclosed subject matter provides a kit comprising a non-spherical biomimetic artificial cell comprising: (a) a three-dimensional microparticle or nanoparticle having an asymmetrical shape; (b) a supported lipid bilayer (SLB); and (c) at least one biomolecule conjugated to the SLB.

In another aspect, the presently disclosed subject matter provides a method for administering a drug to a subject, the method comprising: (a) providing a non-spherical biomimetic artificial cell comprising: (i) a three-dimensional microparticle or nanoparticle having an asymmetrical shape defined by a dimension (a) along an x-axis, a dimension (b) along a y-axis, and a dimension (c) along a z-axis, wherein at least one of (a), (b), or (c) is not equal to at least one other dimension (a), (b), or (c); and (ii) a supported lipid bilayer (SLB) functionalized with a biotin-binding protein or fragment thereof; (b) providing a biotinylated drug; (c) conjugating the biotinylated drug to the biotin-binding protein or fragment thereof on the non-spherical biomimetic artificial cell; and (d) administering the biotinylated drug conjugated to the biotin-binding protein or fragment thereof on the non-spherical biomimetic artificial cell to the subject. In some embodiments, the biotinylated drug is a biotinylated antibody. In some embodiments, the biotinylated antibody is specific for CD28 or the T cell receptor (TCR). In some embodiments, the subject is a human. In some embodiments, the subject is a non-human animal. In some embodiments, administering to the subject comprises administering one or more doses of the biotinylated drug conjugated to the non-spherical biomimetic artificial cell in an amount sufficient to treat a disease, disorder, or dysfunction. In some embodiments, administering is by oral ingestion, through injection, by infusion, through topical administration, through inhalation, through sublingual absorption, through rectal or vaginal delivery, subcutaneously, and combinations thereof.

In certain aspects, the presently disclosed subject matter provides a method for making non-spherical biomimetic artificial cells comprising a biodegradable three-dimensional microparticle or nanoparticle having an asymmetrical shape defined by a dimension (a) along an x-axis, a dimension (b) along a y-axis, and a dimension (c) along a z-axis, wherein at least one of (a), (b), or (c) is not equal to at least one other dimension (a), (b), or (c); a supported lipid bilayer (SLB); and at least one biomolecule conjugated to the SLB, the method comprising: (a) providing or preparing a plurality of three-dimensional microparticles or nanoparticles having an asymmetrical shape; (b) harvesting the plurality of three-dimensional microparticles or nanoparticles having an asymmetrical shape; (c) providing a plurality of liposomes; (d) fusing the plurality of liposomes to the plurality of three-dimensional microparticles or nanoparticles having an asymmetrical shape under sonication to form a plurality of three-dimensional microparticles or nanoparticles having an asymmetrical shape and a supported lipid bilayer (SLB); and (e) conjugating a plurality of biomolecules to the surface of the plurality of three-dimensional microparticles or nanoparticles having an asymmetrical shape and a supported lipid bilayer (SLB) to form the non-spherical biomimetic artificial cells.

In some embodiments, preparing a plurality of three-dimensional microparticles or nanoparticles having an asymmetrical shape comprises: (a) providing or preparing a plurality of microparticles or nanoparticles; (b) preparing a film comprising the plurality of microparticles or nanoparticles; (c) stretching the film comprising the plurality of microparticles or nanoparticles to form a plurality of three-dimensional microparticles or nanoparticles having an asymmetrical shape; and (d) harvesting the plurality of three-dimensional microparticles or nanoparticles having an asymmetrical shape. In some embodiments, the film is heated before being stretched.

In some embodiments, the plurality of biomolecules is a plurality of biotin-binding proteins or fragments thereof, and the method further comprises adding a plurality of maleimide-functionalized lipids into the plurality of liposomes; thereby conjugating a thiolated biotin-binding protein or fragment thereof to the surface of the SLB. In some embodiments, the biotin-binding protein or fragment thereof is selected from the group consisting of avidin, streptavidin, neutravidin, and fragment thereof.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1 shows a representative schematic representation of ellipsoidal and spherical supported lipid bilayer (SLB) synthesis. Non-spherical microparticles are prepared utilizing a thin-film stretching method starting from spherical PLGA microparticles prepared by single emulsion. Liposomes that are approximately 200 nm in size are prepared by extrusion and then sonicated in the presence of microparticles to yield supported lipid bilayers with shape specified by the initial particle template;

FIG. 2A, FIG. 2B, FIG. 2C, FIG. 2D, and FIG. 2E show spherical and non-spherical particles utilized for the synthesis of supported lipid bilayers. SEM images of: (FIG. 2A) spherical and (FIG. 2B) ellipsoidal microparticles utilized as templates for the support of the lipid bilayers; (FIG. 2C) size of spherical particles; and (FIG. 2D and FIG. 2E) measured aspect ratio of spherical and ellipsoidal particles;

FIG. 3A, FIG. 3B, FIG. 3C, FIG. 3D show that non-spherical and spherical lipid bilayers can be synthesized utilizing a pre-formed particle template: confocal micrographs of (FIG. 3A) spherical and (FIG. 3B) ellipsoidal particles encapsulating 7-AMC coated with a fluorescent lipid bilayer; and representative profile of the two fluorescence channels across the (FIG. 3C) center of the sphere and the (FIG. 3D) long axis of the ellipsoid;

FIG. 4A and FIG. 4B show: (FIG. 4A) total protein conjugation amount of fluorescent avidin to spherical and ellipsoidal supported lipid bilayers; and (FIG. 4B) efficiency of conjugation for various ratios of avidin to mass of particles in synthesis. A two way ANOVA was performed to analyze statistical differences in the efficiency data set: p=0.0303 for shape/dose interaction, p=0.0057 for shape impact on results, and p=0.0013 for dose impact on results. There was no significant difference between shapes at any dose tested as evaluated by Bonferroni's post test (p>0.05) except for at 1 μg avidin/mg PLGA (p<0.01);

FIG. 5A, FIG. 5B, FIG. 5C, and FIG. 5D show interchangeable protein surface conjugation to spherical and non-spherical lipid bilayers: (FIG. 5A) spherical and (FIG. 5B) ellipsoidal supported lipid bilayers encapsulating 7-AMC conjugated to avidin-biotin-fluorophore conjugate on the surface; (FIG. 5C) total protein captured by particles exhibits dependency on the amount of protein dosed in synthesis; and (FIG. 5D) efficiency of conjugation between spherical and non-spherical supported lipid bilayers at various doses is similar. Error bars represent SEM of 3 replicates;

FIG. 6A, FIG. 6B, FIG. 6C, and FIG. 6D show that spherical (FIG. 6A and FIG. 6C) and non-spherical (FIG. 6B and FIG. 6D) protein conjugated lipid bilayers are stable before (FIG. 6A and FIG. 6B) and after lyophilization (FIG. 6C and FIG. 6D);

FIG. 7A, FIG. 7B, FIG. 7C, FIG. 7D, FIG. 7E, FIG. 7F, FIG. 7G, FIG. 7H, FIG. 7I, and FIG. 7J show: (FIG. 8A) spherical and (FIG. 8B) ellipsoidal SLBs with Cy5 biotin conjugated to the surface were prepared and incubated 25° C. and 37° C. over the course of 7 days. Confocal image analysis of spherical particles incubated at (FIG. 8C) 25° C. and (FIG. 8D) 37° C. and ellipsoidal particles incubated at (FIG. 8E) 25° C. and (FIG. 8F) 37° C. after 1 day reveal the supported lipid bilayers remain intact. Subsequent confocal image analysis of spherical particles incubated at (FIG. 8G) 25° C. and (FIG. 8H) 37° C. and ellipsoidal particles incubated at (FIG. 8I) 25° C. and (FIG. 8J) 37° C. after 7 days revealed the supported lipid bilayers remained stable for a week despite slight shape retraction of the ellipsoidal particles;

FIG. 8A, FIG. 8B, and FIG. 8C show that supported lipid bilayers bearing conjugated proteins demonstrate fluidity: (FIG. 8A) spherical SLBs and (FIG. 8B) ellipsoidal SLBs were subjected to region specific bleaching under confocal microscopy and then subsequently imaged to measure recovery of fluorescence; and (FIG. 8C) from an exponential fit of the recovery of the lipid bilayers, lateral diffusion constants were derived for the spherical and ellipsoidal supported lipid bilayers and were determined to be equivalent;

FIG. 9 shows that the viability of macrophages during cell uptake experiments is essentially unaltered by the presence of lipid coated particles. Macrophages were incubated for 4 hours with the indicated concentration of particles and viability was established by cell titer assay and normalization to the untreated control. Error bars represent standard deviation of 4 replicates. A two way ANOVA was performed to analyze statistical differences in the data set: p=0.0633 for shape/dose interaction, p=0.1495 for shape impact on results, and p<0.0001 for dose impact on results. There was no significant difference between shapes at any dose tested as evaluated by Bonferroni's post test (p>0.05) ; and

FIG. 10A, FIG. 10B, and FIG. 10C show that macrophage uptake is shape dependent for spherical and ellipsoidal supported lipid bilayers. Confocal micrographs of non-specific uptake of (FIG. 10A) spherical and (FIG. 10B) ellipsoidal supported lipid bilayers by macrophages demonstrates that ellipsoids resist non-specific cell uptake. Flow cytometry of macrophages (FIG. 10C) treated with supported lipid bilayers of spherical and ellipsoidal shape reinforce the conclusion that ellipsoidal supported lipid bilayers resist cellular uptake.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

In some embodiments, the presently disclosed subject matter provides compositions and methods for generating an anisotropic, non-spherical particle with a supported lipid bilayer (SLB) and at least one biomolecule attached to its surface to synergize the benefits of a biomimetic, fluidic surface of a supported lipid bilayer with the benefits of a non-spherical particle. The presently disclosed biomimetic artificial cell maintains the advantageous properties of both lipid polymer hybrid particles and non-spherical particles in terms of stability, biomimetic membrane fluidity, and resistance of non-specific phagocytosis by macrophages. In addition, the presently disclosed biomimetic artificial cell has the ability for modular protein conjugation to the particle surface using versatile bioorthogonal ligation reactions. As used herein, the term “modular protein conjugation” refers to the conjugation of different types of proteins to the particle surface in a standardized way. As used herein, the term “bioorthogonal ligation reactions” refer to ligation reactions of biomolecules to the particle surface that occur independently of each other. In particular embodiments, the anisotropic, non-spherical particle is an ellipsoid. In other particular embodiments, the anisotropic, non-spherical particle is biodegradable.

The presently disclosed subject matter is believed to be the first instance of a multiscale design to mimic on an acellular platform the most critical features of cell-cell interactions including paracrine release of soluble mediators, dynamic radius of curvature, and fluidity of attached molecules on the surface for receptor-mimetic clustering. Compared to designs known in the art for SLB based cellular mimicry, which can emulate one or two of these features, the presently disclosed platform can mimic all three in a single acellular particulate system. Additionally, the presently disclosed biomimetic cells are capable of being functionalized, such as by biotinylated entities, for active targeting mediated drug delivery for use in biomedical therapeutics.

### I. Anisotropic Supported Lipid Bilayers on Biodegradable Micro and Nanoparticles Bearing Biomolecules

In some embodiments, the presently disclosed subject matter provides a non-spherical biomimetic artificial cell comprising: (a) a three-dimensional microparticle or nanoparticle having an asymmetrical shape; (b) a supported lipid bilayer (SLB); and (c) at least one biomolecule conjugated to the SLB. In a preferred embodiment, the artificial cell has an ellipsoidal shape, such as prolate or oblate.

As used herein, by “biomimetic artificial cell,” it is meant an artificial cell that has properties that mimic a natural cell. For example, the presently disclosed biomimetic artificial cells comprise lipid bilayers, which are polar membranes made of layers of lipid molecules. In a natural cell, the cell membrane usually contains lipid bilayers, which aid in the regulation of molecules through the cell membrane. A “supported lipid bilayer” as used herein is a lipid bilayer that sits on a solid support, such as a three-dimensional microparticle or nanoparticle having an asymmetrical shape. As used herein, the term “molecule” generally refers to two or more atoms held together by covalent bonds. Therefore, a molecule can be relatively small, such as the size of a peptide, or it can be relatively large, such as the size of a protein comprising several polypeptides. As used herein, a “molecule” is not restricted by size. As used herein, the term “biomolecule” generally refers to an organic molecule comprising carbon, hydrogen, nitrogen and/or oxygen. Examples include proteins, polysaccharides, nucleic acids, amino acids, DNA, RNA, and the like.

In other embodiments, the presently disclosed subject matter provides a non-spherical biomimetic artificial cell comprising: (a) a three-dimensional microparticle or nanoparticle having an asymmetrical shape, wherein the asymmetrical shape has at least one surface having a radius of curvature along at least one axis selected from one of the following ranges: (a) about 1 nm to about 10 nm; (b) about 11 nm to about 100 nm; (c) about 101 nm to about 400 nm; (d) about 401 nm to about 1 μm; (e) about 10 μm to about 20 μm; (0 about 20 μm to about 100 μm; and (g) about 101 μm to about 1 mm; (b) a supported lipid bilayer (SLB); and (c) at least one biomolecule conjugated to the SLB.

In yet other embodiments, the presently disclosed subject matter provides a non-spherical biomimetic artificial cell comprising: (a) a three-dimensional microparticle or nanoparticle having an asymmetrical shape defined by a dimension (a) along an x-axis, a dimension (b) along a y-axis, and a dimension (c) along a z-axis, wherein at least one of (a), (b), or (c) is not equal to at least one other dimension (a), (b), or (c); (b) a supported lipid bilayer (SLB); and (c) at least one biomolecule conjugated to the SLB.

As the particle becomes flatter, the radius of curvature becomes larger. Conversely, as a surface on the particle becomes more curved, the radius of curvature becomes smaller. In some embodiments, the particle has at least one surface that has a radius of curvature that does not include the range from about 1 micron to about 10 microns.

In some embodiments, the non-spherical shape comprises a prolate ellipsoid, which is defined by the equation a>b=c. In other embodiments, the non-spherical shape comprises a tri-axial ellipsoid, which can be described by the equation a>b>c. In yet other embodiments, the non-spherical shape comprises an oblate ellipsoid, which can be described by the equation a=b>c. In other embodiments, the non-spherical shape has a dimension (a) along the x axis is equal to the dimension (b) along they axis, both of which are much less than dimension (c) along the z-axis, such that a=b<<c and the three-dimensional microparticle or nanoparticle comprises a rod.

As used herein, the term “nanoparticle,” refers to a particle having at least one dimension in the range of about 1 nm to about 1000 nm, including any integer value between 1 nm and 1000 nm (including about 1, 2, 5, 10, 20, 50, 60, 70, 80, 90, 100, 200, 500, and 1000 nm and all integers and fractional integers in between). In some embodiments, the nanoparticle has at least one dimension, e.g., a diameter, of about 100 nm. In some embodiments, the nanoparticle has a diameter of about 200 nm. In other embodiments, the nanoparticle has a diameter of about 500 nm. In yet other embodiments, the nanoparticle has a diameter of about 1000 nm (1 μm). In such embodiments, the particle also can be referred to as a “microparticle.” Thus, the term “microparticle” includes particles having at least one dimension in the range of about one micrometer (μm), i.e., 1×10⁻⁶ meters, to about 1000 μm. The term “particle” as used herein is meant to include nanoparticles and microparticles.

In some embodiments, the presently disclosed subject matter provides a non-spherical biomimetic artificial cell, wherein the three-dimensional microparticle or nanoparticle comprises an ellipsoid selected from the group consisting of: a prolate ellipsoid, wherein the dimension (a) along the x-axis is greater than the dimension (b) along the y-axis, and wherein the dimension (b) along the y-axis is equal to the dimension (c) along the z-axis, such that the prolate ellipsoid can be described by the equation a>b=c; a tri-axial ellipsoid, wherein the dimension (a) along the x-axis is greater than the dimension (b) along the y-axis, and wherein the dimension (b) along the y-axis is greater than the dimension (c) along the z-axis, such that the tri-axial ellipsoid can be described by the equation a>b>c; and an oblate ellipsoid, wherein the dimension (a) along the x-axis is equal to the dimension (b) along the y-axis, and wherein the dimension (b) along the y-axis is greater than the dimension (c) along the z-axis, such that the oblate ellipsoid can be described by the equation a=b>c. The presently disclosed asymmetrical particles, however, do not include embodiments in which a=b=c.

In some embodiments, the microparticle or nanoparticle has an aspect ratio ranging from about 1.1 to about 5. In other embodiments, the aspect ratio has a range from about 5 to about 10. In some embodiments, the aspect ratio has a range from about 1.5 to about 3.5, including 1.5, 2, 2.5, 3, and 3.5.

Generally, the three-dimensional microparticle or nanoparticle comprises a material having one or more of the following characteristics: (i) one or more degradable linkages; (ii) a stretchable Young's modulus ranging from 10⁶-10¹⁰ N/m² and in some embodiments 10⁷-10⁹ N/m²; and (iii) a glass transition temperature such that the material comprising the three-dimensional microparticle or nanoparticle is a solid at room temperature and/or body temperature. The particles also can be composed of copolymers, with one or more constituents being defined as above.

As used herein, “glass transition temperature” refers to the temperature at which amorphous polymers undergo a transition from a rubbery, viscous amorphous liquid, to a brittle, glassy amorphous solid. As used herein, “Young's modulus of elasticity” quantifies the elasticity of the polymer. It is defined, for small strains, as the ratio of rate of change of stress to strain.

As used herein, “biodegradable” compounds are those that, when introduced into cells, are broken down by the cellular machinery or by hydrolysis into components that the cells can either reuse or dispose of without significant toxic effect on the cells (i.e., fewer than about 20% of the cells are killed when the components are added to cells in vitro). The components preferably do not induce inflammation or other adverse effects in vivo. In certain preferred embodiments, the chemical reactions relied upon to break down the biodegradable compounds are uncatalyzed.

Generally, to be biodegradable, the presently disclosed materials, e.g., microparticles and/or nanoparticles, contain a degradable linkage. Representative degradable linkages include, but are not limited to:

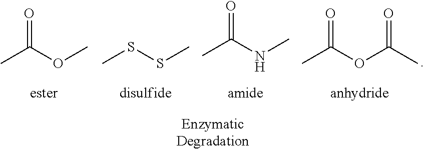

In some embodiments, the three-dimensional microparticle or nanoparticle comprises a material having one or more of the following characteristics: (i) one or more degradable linkages; (ii) a stretchable modulus; and (iii) a glass transition temperature such that the material comprising the three-dimensional microparticle or nanoparticle is a solid at room temperature and/or body temperature. In other embodiments, the degradable linkage is selected from the group consisting of an ester linkage, a disulfide linkage, an amide linkage, an anhydride linkage, and a linkage susceptible to enzymatic degradation. In particular embodiments, the microparticle or nanoparticle comprises a biodegradable polymer or blends of polymers selected from the group consisting of poly(lactic-co-glycolic acid) (PLGA), poly(beta-amino ester) (PBAE), polycaprolactone (PCL), polyglycolic acid (PGA), polylactic acid (PLA), poly(acrylic acid) (PAA), poly-3-hydroxybutyrate (P3HB) and poly(hydroxybutyrate-co-hydroxyvalerate). In other embodiments, nondegradable polymers that are used in the art, such as polystyrene, are blended with a degradable polymer or polymers from above to create a copolymer system. Accordingly, in some embodiments, the microparticle or nanoparticle further comprises a biodegradable polymer or blends of polymers blended with a nondegradable polymer.

Other biodegradable polymers suitable for use with the presently disclosed subject matter are provided in International PCT Patent Application Publication No. WO/2010/132879 for “Multicomponent Degradable Cationic Polymers,” to Green et al., published Nov. 18, 2010, which is incorporated herein by reference in its entirety.

In some embodiments, at least one biomolecule conjugated to the SLB comprises at least one entity selected from the group consisting of a drug, therapeutic agent, protein, peptide, sugar, and polysaccharide. In some embodiments, at least one biomolecule is a protein or a fragment thereof. As used herein, a “drug” is a substance that has a physiological effect when introduced into a subject. A drug also can be tested in vitro, for example, in cell culture, to determine its effect on a cell. A “protein drug” is a drug comprising a peptide(s) or polypeptide(s).

As used herein, a “peptide”, “protein”, or “protein or fragment thereof” comprises a string of at least three amino acids linked together by peptide bonds. The terms “protein” and “peptide” may be used interchangeably. Peptide may refer to an individual peptide or a collection of peptides. Also, one or more of the amino acids in a presently disclosed peptide may be modified, for example, by the addition of a chemical entity, such as a carbohydrate group, a phosphate group, a farnesyl group, an isofarnesyl group, a fatty acid group, a linker for conjugation, functionalization, or other modification, and the like.

As used herein, a “carbohydrate” is a biological molecule comprising carbon (C), hydrogen (H) and oxygen (O) atoms, usually with a hydrogen:oxygen atom ratio of 2:1. As used herein, “polysaccharides” are polymeric carbohydrate molecules composed of long chains of monosaccharide units bound together by glycosidic linkages. They range in structure from linear to highly branched. Some examples include storage polysaccharides such as starch and glycogen, and structural polysaccharides such as cellulose and chitin. As used herein, the term “sugar” refers to short-chain, soluble carbohydrates, many of which are used in food.

In some embodiments, the non-spherical biomimetic artificial cell comprises at least biomolecule on one or more surfaces of the non-spherical biomimetic artificial cell and/or within the non-spherical biomimetic artificial cell. In other embodiments, at least one biomolecule is found on the surface of the artificial cell. In still other embodiments, at least one biomolecule conjugated to the SLB is found on the surface of the SLB.

Biotin has been found to have a high affinity for biotin-binding proteins, such as avidin, streptavidin, and neutravidin. Biotinylation is the process of covalently attaching biotin to a protein, antibody, enzyme, nucleic acid or other molecule. Biotinylated molecules can comprise multiple biotin molecules. In some embodiments, these biotinylated molecules can bind to biotin-binding proteins on the presently disclosed non-spherical artificial cells. Accordingly, in some embodiments, the protein or fragment thereof is a biotin-binding protein or fragment thereof. In other embodiments, the biotin-binding protein or fragment thereof is selected from the group consisting of avidin, streptavidin, neutravidin, and fragment thereof.

In some embodiments, the presently disclosed non-spherical artificial cells are capable of interacting with a T cell receptor (TCR). In other embodiments, the presently disclosed non-spherical biomimetic artificial cell mimics the function of a biological antigen presenting cell. A T cell or T lymphocyte is a cell that belongs to a group of white blood cells known as lymphocytes and plays a central role in cell-mediated immunity. Different types of T cells include, but are not limited to, T helper cells, cytotoxic T cells, memory T cells, regulatory T cells (also known as suppressor cells), and natural killer T cells. A T cell can be distinguished from other lymphocytes by the presence of a T cell receptor on its cell surface. A T cell receptor is a protein that is found on the surface of a T cell and it is responsible for recognizing antigens bound to MHC molecules. This recognition ensures that only a T cell with a TCR specific to a particular antigen is activated. In some embodiments, the interaction of the TCR with a MHC:antigen complex is the first signal in the activation or modulation of a T cell. The antigen can be presented to the T cell by a MHC-dimer or -tetramer molecule. The MHC-dimer or -tetramer molecule can be easily loaded with any MHC-restricted peptide of interest. By loaded, it is meant that the peptide is attached in some way to the MHC-dimer or -tetramer, whether by covalent interactions or by noncovalent interactions or both.

In some embodiments, the molecule capable of interacting with the TCR is a peptide. In other embodiments, peptides interacting with MHC Class II molecules are typically 13 to 17 amino acids in length, and in still other embodiments, shorter or longer peptides are common and allowed. In further embodiments, peptides interacting with MHC Class I molecules have more stringent requirements of generally less than 15 amino acids length.

In some embodiments, the peptide is loaded onto a MHC-Ig molecule or a HLA:Ig molecule before interacting with the TCR. In other embodiments, the HLA:Ig molecule is a HLA:A2:Ig molecule.

An important feature of the SLB is the capability to mimic natural membrane fluidity. It has been shown herein below that the presently disclosed non-spherical biomimetic artificial cells comprise lipid bilayers that have similar diffusion constants and recovery time as spherical SLBs and proteins on natural, biological membranes. Accordingly, in some embodiments, the SLB retains lateral diffusive properties as compared to the SLB of a similar spherical biomimetic artificial cell. In other embodiments, the membrane fluidity is selected from the group consisting of 10e-7 to 10e-8 cm²/s, in some embodiments, 10e-8 to 10e-9 cm²/s, in other embodiments, 10e-9 to 10e-10 cm²/s, in yet other embodiments, 10e-10 to 10e-11 cm²/s, and in still yet other embodiments, 10e-11 to 10e-12 cm²/s.

The terms “lateral diffusive properties” or “lateral membrane fluidity” refer to the ability of the lipids of the SLB, and any proteins conjugated to the lipids, to move laterally in the SLB as is found in a natural lipid bilayer.

Another important feature of the presently disclosed non-spherical artificial cells is that they are more resistant to phagocytosis as compared to a similar spherical artificial cell. In some embodiments, the presently disclosed artificial cells are at least 10% (e.g., 20%, 30%, 40%, or more) more resistant to phagocytosis than a similar spherical artificial cell. As used herein, the term “phagocytosis” refers to the process by which a cell engulfs a solid particle to form an internal vesicle known as a phagosome.

In some embodiments, the presently disclosed subject matter provides a kit comprising a presently disclosed non-spherical biomimetic artificial cell. In some embodiments, the kits comprise non-spherical biomimetic artificial cells in an amount sufficient to administer a drug to a subject at least one time. Typically, the non-spherical biomimetic artificial cells of the kit will be supplied in one or more container, each container containing a sufficient amount of artificial cells for at least one dosing of the subject. In other embodiments, the presently disclosed subject matter includes a kit comprising the raw materials for making the presently disclosed non-spherical biomimetic artificial cells.

### II. Methods for Using Anisotropic Supported Lipid Bilayers on Biodegradable Micro and Nanoparticles

The presently disclosed subject matter also provides methods for using the presently disclosed non-spherical biomimetic artificial cells. The presently disclosed subject matter allows for more accurate mimicry of natural cells through the presentation of laterally mobile proteins on the surface of anisotropic biodegradable particles. The presently disclosed methods for using a presently disclosed non-spherical biomimetic artificial cell can be in vitro, in vivo, or ex vivo. Examples of its use include generating anisotropic supported lipid bilayer based artificial antigen presenting cells, targeted drug delivery, and the like.

In some embodiments, the presently disclosed subject matter provides a method for administering a drug to a subject, the method comprising: (a) providing a non-spherical biomimetic artificial cell comprising: (i) a three-dimensional microparticle or nanoparticle having an asymmetrical shape defined by a dimension (a) along an x-axis, a dimension (b) along a y-axis, and a dimension (c) along a z-axis, wherein at least one of (a), (b), or (c) is not equal to at least one other dimension (a), (b), or (c); and (ii) a supported lipid bilayer (SLB) functionalized with a biotin-binding protein or fragment thereof; (b) providing a biotinylated drug; (c) conjugating the biotinylated drug to the biotin-binding protein or fragment thereof on the non-spherical biomimetic artificial cell; and (d) administering the biotinylated drug conjugated to the biotin-binding protein or fragment thereof on the non-spherical biomimetic artificial cell to the subject. As used herein, the term “conjugate” refers to a binding or joining of two molecules, whether covalently or noncovalently.

In some embodiments, the biotinylated drug is a biotinylated antibody. In other embodiments, the biotinylated antibody is specific for a receptor on a biological cells surface or emulates a biological cell ligand. This emulation can include ligands that mimic or bind to targets on immune cells (CD45, CD8, CD4, CD25, Foxp3, CD3, CD20, CD24, CD16, CD40, CD56, CD137), stem cells (CD34), cancer cells including putative cancer stem cells (CD 133, CXCR4), endothelial cells (CD31), and other differentiated or non-differentiated cells known to one in the art. In still other embodiments, the biotinylated antibody is specific for CD28 or the T cell receptor (TCR). As used herein, the term “antibody” refers to a polypeptide or group of polypeptides, which are comprised of at least one binding domain, where an antibody binding domain is formed from the folding of variable domains of an antibody molecule to form three-dimensional binding spaces with an internal surface shape and charge distribution complementary to the features of an antigenic determinant of an antigen, which allows an immunological reaction with the antigen. Antibodies include recombinant proteins comprising the binding domains, as wells as fragments, including Fab, Fab′, F(ab)2, and F(ab′)2 fragments.

In some embodiments, administering to the subject comprises administering one or more doses of the biotinylated drug conjugated to the non-spherical biomimetic artificial cell in an amount sufficient to treat a disease, disorder, or dysfunction. A disease, disorder, or dysfunction refers to any condition that affects the health of a subject. As the presently disclosed subject matter can be used for targeting a wide variety of drugs, methods and compositions of the presently disclosed subject matter will affect a wide variety of diseases, disorders, or dysfunctions. Examples include, but are not limited to, cancer and infectious diseases. The presently disclosed subject methods and compositions may be modified to treat a particular disease, disorder, or dysfunction. In other embodiments, administering is by oral ingestion, through injection, by infusion, through topical administration, through inhalation, through sublingual absorption, through rectal or vaginal delivery, subcutaneously, and combinations thereof.

In general, the “effective amount” of an active agent refers to the amount necessary to elicit the desired biological response. As will be appreciated by those of ordinary skill in this art, the effective amount of an agent may vary depending on such factors as the desired biological endpoint, the agent to be delivered, the composition of the encapsulating matrix, the target tissue, and the like. As used herein, a “dose” refers to the amount of non-spherical biomimetic artificial cells administered to a subject that is sufficient to treat the subject for a disease, disorder, or dysfunction.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. Accordingly, in some embodiments, the subject is a human. In other embodiments, the subject is a non-human animal.

### III. Methods for Preparing Anisotropic Supported Lipid Bilayers on Biodegradable Micro and Nanoparticles

The presently disclosed subject matter also provides methods for making non-spherical biomimetic artificial cells. In some embodiments, the presently disclosed subject matter provides a method for making non-spherical biomimetic artificial cells comprising a biodegradable three-dimensional microparticle or nanoparticle having an asymmetrical shape defined by a dimension (a) along an x-axis, a dimension (b) along a y-axis, and a dimension (c) along a z-axis, wherein at least one of (a), (b), or (c) is not equal to at least one other dimension (a), (b), or (c); a supported lipid bilayer (SLB); and at least one biomolecule conjugated to the SLB, the method comprising: (a) providing or preparing a plurality of three-dimensional microparticles or nanoparticles having an asymmetrical shape; (b) harvesting the plurality of three-dimensional microparticles or nanoparticles having an asymmetrical shape; (c) providing a plurality of liposomes; (d) fusing the plurality of liposomes to the plurality of three-dimensional microparticles or nanoparticles having an asymmetrical shape under sonication to form a plurality of three-dimensional microparticles or nanoparticles having an asymmetrical shape and a supported lipid bilayer (SLB); and (e) conjugating a plurality of biomolecules to the surface of the plurality of three-dimensional microparticles or nanoparticles having an asymmetrical shape and a supported lipid bilayer (SLB) to form the non-spherical biomimetic artificial cells.

In other embodiments, the presently disclosed subject matter provides a method for preparing a plurality of three-dimensional microparticles or nanoparticles having an asymmetrical shape, the method comprising: (a) providing or preparing a plurality of microparticles or nanoparticles; (b) preparing a film comprising the plurality of microparticles or nanoparticles; (c) stretching the film comprising the plurality of microparticles or nanoparticles to form a plurality of three-dimensional microparticles or nanoparticles having an asymmetrical shape; and (d) harvesting the plurality of three-dimensional microparticles or nanoparticles having an asymmetrical shape. In other embodiments, the film is heated before being stretched.

In other embodiments, the plurality of biomolecules is a plurality of biotin-binding proteins or fragments thereof, and the method further comprises adding a plurality of maleimide-functionalized lipids into the plurality of liposomes; thereby conjugating a thiolated biotin-binding protein or fragment thereof to the surface of the SLB.

In still other embodiments, the biotin-binding protein or fragment thereof is selected from the group consisting of avidin, streptavidin, neutravidin, and fragments thereof.

In further embodiments, it has previously been found that hyper-dense ligand coated particles that have a surface density greater than what has currently been achieved can be formed. These particles can be formed by stretching microparticles or nanoparticles into an asymmetrical shape, adding the functional ligands to the particles, and then allowing the particles to relax back partially while still keeping an asymmetrical shape. For example, a plurality of three-dimensional microparticles or nanoparticles can be relaxed back partially or completely to a near spherical shape. In the case of an ellipsoid, the parameters (a), (b), or (c) are approximately equal in a “near spherical shape.”

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The following Examples are offered by way of illustration and not by way of limitation.

### Example 1

### Materials

Particle Preparation and Characterization: Acid terminated poly(lactic-co-glycolic acid) (PLGA-85:15 L:G ratio, MW 45,000 Da—55,000 Da) (Akina Inc.; West Lafayette, Ind.) was dissolved in 5 mL of dichloromethane (DCM) at a concentration of 20 mg/mL. In order to visualize particles under fluorescence microscopy, 7-amino-4-methyl coumarin (7-AMC-Sigma-Aldrich; St. Louis, Mo.) or Nile Red (Life Technologies; Grand Island, N.Y.) were added to the DCM solution at a 1% w/w ratio to the polymer. The resulting solution was homogenized by a T-25 digital ULTRA-TURRAX IKA tissue homogenizer at 5,000 rpm for 1 min in 50 mL of 1% PVA solution. The subsequent emulsion was then transferred to 100 mL of 0.5% PVA solution agitated by magnetic stir bar and the DCM was allowed to evaporate over the course of 4 hrs. The suspended particles were centrifuged out of solution at 3000 g for 5 min and washed 3 times with water. The resulting particles were flash frozen in liquid nitrogen and lyophilized prior to use.

To synthesize non-spherical ellipsoidal particles, the thin film stretching method developed by Ho et al. (1993) was utilized which was recently automated (Meyer, Meyer, and Green, 2015) to generate ellipsoidal microparticles to serve as the template for the SLBs. Spherical particles synthesized by emulsion were cast into a thin film of 10% poly(vinyl alcohol) and 2% glycerol at a concentration of 5 mg/mL and 10 mL of this solution was deposited into a rectangular petri dish. The film was allowed to dry overnight, and the next day the film was cut to size and mounted onto an automated thin film stretching device. The entire apparatus was heated up to 90° C. and the film was stretched 2 fold in one direction to produce ellipsoidal particles with a major axis roughly 2 fold times the original particle diameter and a minor axis roughly 0.7 times the original particle diameter. After stretching, the film was allowed to cool back down to room temperature and then was dissolved in water. Particles were washed and subsequently lyophilized prior to use and characterization.

Particle characterization was conducted using scanning electron microscopy (Leo FESEM). Lyophilized particles were mounted onto an aluminum tack (Electron Microscopy Services; Hatfield, Pa.) using carbon tape (Nisshin EM Co.; Tokyo, Japan). The particles were then sputter coated with 30 nm of gold-palladium alloy. After sputter coating, the particles were imaged by SEM. Particle size and aspect ratio data were obtained by ImageJ analysis of the subsequent SEM images.

Supported Lipid Bilayer Preparation and Imaging: Non-spherical supported lipid bilayers (SLBs) were prepared utilizing a two-step method similar in concept to what has previously described for spherical particles (Ashley et al., 2011). 1,2-dioleoyl-sn-glycero-3-phosphocholine (DOPC) and cholesterol (Avanti Polar Lipids; Alabaster, Ala.) were mixed into a 70:30 w/w ratio. For fluorescent lipid imaging studies, rhodamine conjugated DOPC (Avanti Polar Lipids; Alabaster, Ala.) was mixed with cholesterol in a 70:30 w/w ratio. For surface functionalization, 1,2-dioleoyl-sn-glycero-3-phosphoethanolamine-N-[4-(p-maleimidomethyl)cyclohexane-carboxamide] (MCC-DOPC) (Avanti Polar Lipids; Alabaster, Ala.), DOPC, and cholesterol were mixed in a 35:35:30 w/w ratio. 1 mg total of the lipids was aliquoted and left to dry into a thin film overnight under a vacuum. The lipids were then hydrated in 1 mL of water. The lipids were heated to 60° C. and extruded through a 200 nm filter (Avanti Polar Lipids; Alabaster, Ala.). 200 nm liposome formation was verified with dynamic light scattering (Malvern Instruments; Westborough, Mass.). The liposomes were then mixed with spherical or non-spherical particles (in a 33.4 μg liposome to 1 mg particle ratio) and sonicated for 30 s at 2 W power in a 1.5 mL Eppendorf tube. Temperature was maintained at 4° C. with an aluminum cooling block (Light Labs; Dallas, Tex.). The subsequent supported lipid bilayers were purified from solution through centrifugation at 4° C. for 5 min at 300 g. After three washes, the supported lipid bilayers were stored at 4° C. until further use.

To analyze the formation of SLBs, confocal imaging of PLGA particles encapsulating 7-AMC were coated with rhodamine lipid containing liposomes. Confocal image acquisition was completed with a Zeiss 780 FCS Confocal Microscope. To derive profile information, the ImageJ profile measurement tool was used and a line was drawn through the particle to determine relative fluorescence information.

Surface Protein Conjugation and Characterization: In order to functionalize the supported lipid bilayers to be receptive to any form of protein conjugation, the surface was first functionalized with thiolated avidin (Protein Mods; Madison, Wis.). To determine whether or not the avidin could conjugate to the surface of the maleimide activated particle, biotinylated fluorescein (Sigma-Aldrich; St. Louis, Mo.) was pre-conjugated with the avidin and then dialyzed overnight with a 10 kDa MWCO dialysis bag (Life Technologies; Grand Island, N.Y.). The particles were then reacted overnight with various amounts of fluorescent avidin and washed three times. Fluorescence intensity was measured under a plate reader and correlated to the amount of fluorescent avidin on the surface of the particle.

To evaluate the capabilities to conjugate a target biotinylated protein to the surface of the supported lipid bilayers, the SLBs of ellipsoidal and spherical shape were formed and conjugated to the thiolated avidin overnight at 4° C. at a 4 μg avidin/mg PLGA ratio. Then, Cy5-biotin (Click Chemistry Tools; Scottsdale, Ariz.) was conjugated at a concentration of 4 μg Cy5-biotin/mg PLGA ratio for 1 hour at room temperature. After washing 3 times at 4° C., the conjugation was evaluated through confocal imaging of the particles.

To confirm that this method would work for a bioactive protein, biotinylated anti CD28 was utilized as a model protein for particle surface capture. Avidin functionalized SLBs were prepared as previously, but instead of a fluorophore, the protein was added in at various concentrations to test reaction efficiency. To quantitate the amount of protein bound to the surface, the particles were washed 3 times, the supernatants were collected, and the supernatants were analyzed for a reduction in protein content utilizing an Octet Red system (Forte Bio; Menlo Park, Calif.). Reduction in protein content in the supernatant was then converted to protein immobilized on the surface through subtraction from the total amount of protein added into the system.

FRAP Analysis: In order to confirm the fluidic character of the spherical and ellipsoidal supported lipid bilayers, diffusivity was evaluated utilizing the fluorescence recovery after photobleaching technique (FRAP). Particles were suspended in 1× PBS and incubated at 37° C. for the duration of the FRAP experiments. Using a Zeiss 780 FCS Confocal Microscope, rectangular regions of interest were selected on a number of particles, these regions were bleached, following which the fluorescent recovery in the selected regions was tracked over time. The fluorescent intensity measured at each time point (I(t)) was then converted to a normalized fluorescent intensity (NFI(t)) using the following equation:

$$NFI ( t ) = \frac{I ( t ) - {I}_{post bleach}}{{I}_{pre bleach} * {I}_{post recovery}}$$

The NFI was then plotted against time and fit to a one phase exponential association curve using GraphPad Prism 6 (GraphPad Software, Inc.; La Jolla, Calif.). From the fit of the curves, time constants for half recovery were derived (t_{0.5}). In order to determine diffusion constants, a circular bleaching region of the particle was assumed with a radius determined from Image J analysis of the 2D images of post bleached particles (r_{bleach}). These values were then applied to the model set forth by Kang et. al.(2012) for derivation of lipid diffusion constants from FRAP:

$${D}_{lipid} = \frac{{r}_{bleach}^{2}}{4 * {t}_{0 , 5}}$$

Diffusion constants for 10 particles for both shapes were computed with this model and compared.

Cellular Uptake Studies: RAW 264.7 (ATCC) macrophages were cultured in T-175 flasks in Dulbecco's Minimal Essential Media (Life Technologies; Grand Island, N.Y.) supplemented with 10% fetal bovine serum and penicillin/streptomycin antibiotics. For flow cytometry studies, cells were harvested through the use of a cell scraper and seeded into 24-well plates at a density of 75,000 cells/well. After cell adherence, the cells were stained with Vybrant CFDA-SE Cell Tracer Kit following the manufacturer's protocol (Life Technologies; Grand Island, N.Y.) as a counterstain to identify live cells in flow cytometry. The cell media was removed and replaced with 500 μL of cell media containing either spherical or ellipsoidal particles encapsulating 5(6)-carboxy-tetramethyl-rhodamine (Merck KGaA; Darmstadt, GE) in a 2-fold dilution series starting at 0.5 mg particles/mL. The cells were then incubated for 4 hours and dissociated from the plate by vigorous tituration prior to analysis by flow cytometry. Cell viability was evaluated using a cell titer kit (Promega; Madison, Wis.) following the manufacturer's protocol.

For confocal imaging, cells were seeded at the same concentration and incubated with the same amount of fluorescently labeled particles, except the incubation was conducted in LabTek Chamber slides (Fisher Scientific; Pittsburgh, Pa.). The cells were washed 3x with PBS and then fixed in 10% formalin stabilized with methanol for 15 minutes (Sigma-Aldrich; St. Louis, Mo.). After fixing, actin was stained with Alexa 488 Phalloidin (Life Technologies; Grand Island, N.Y.) and the nucleus was visualized with the DAPI stain, both following the manufacturer's protocol. The cells were then visualized using confocal microscopy on a Zeiss 780 FCS.

### Example 2

### Procedure to Generate Non-Spherical, Ellipsoidal SLB with a Biodegradable Polymer Support

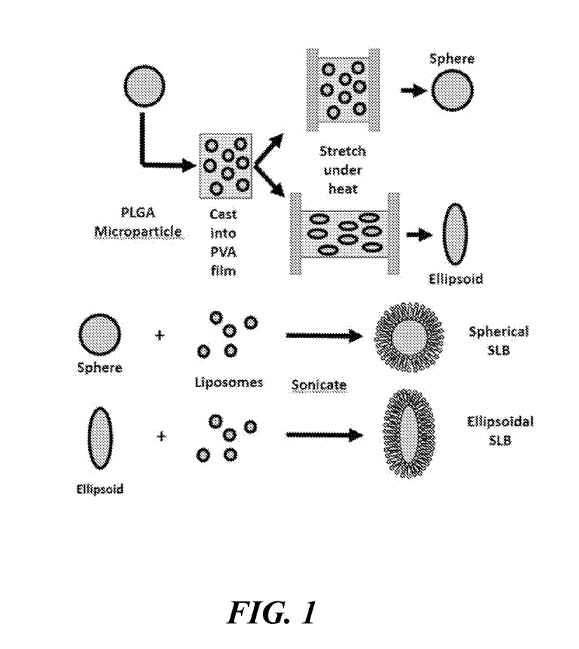

Using the thin-film stretching method developed by Ho et. al. (1993), recently expanded to produce polymeric particles of a wide variety of shapes (Champion et al., 2007), ellipsoidal microparticles are first generated to serve as the template for the SLBs. (FIG. 1). Then, preformed 200-nm liposomes are fused to the ellipsoidal particles under sonication to generate the final ellipsoidal SLB.

A device suitable for use in stretching the presently disclosed films comprising nanoparticles and microparticles is disclosed in International PCT Patent Application Publication No. WO/2013/086500 for “Artificial Antigen Presenting Cells Having a Defined And Dynamic Shape,” to Green et al., published Jun. 13, 2013, which is incorporated herein by reference in its entirety.

### Example 3

### Results

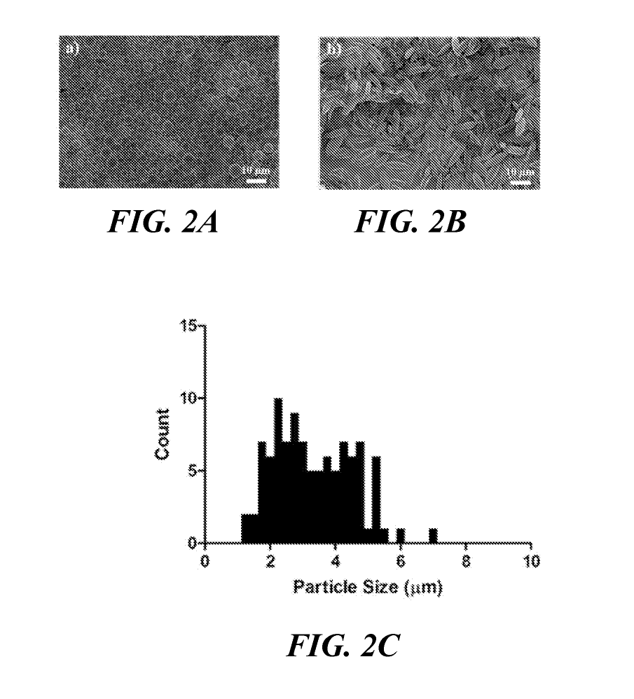

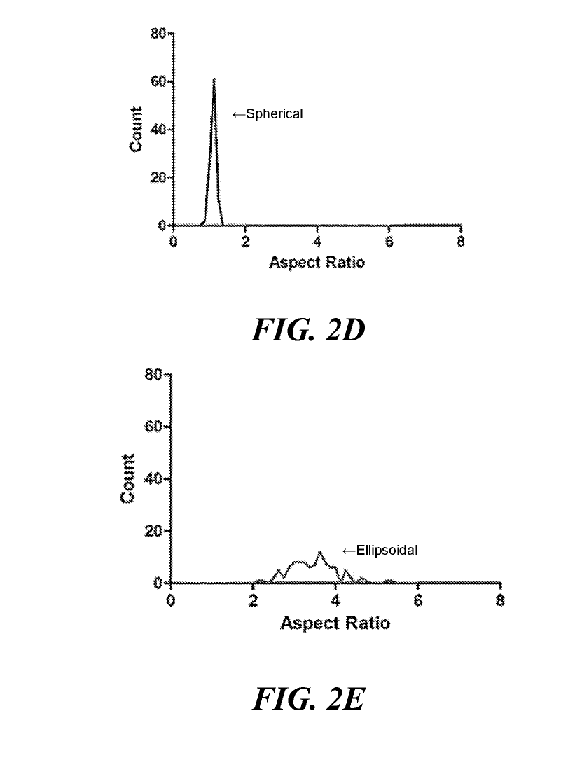

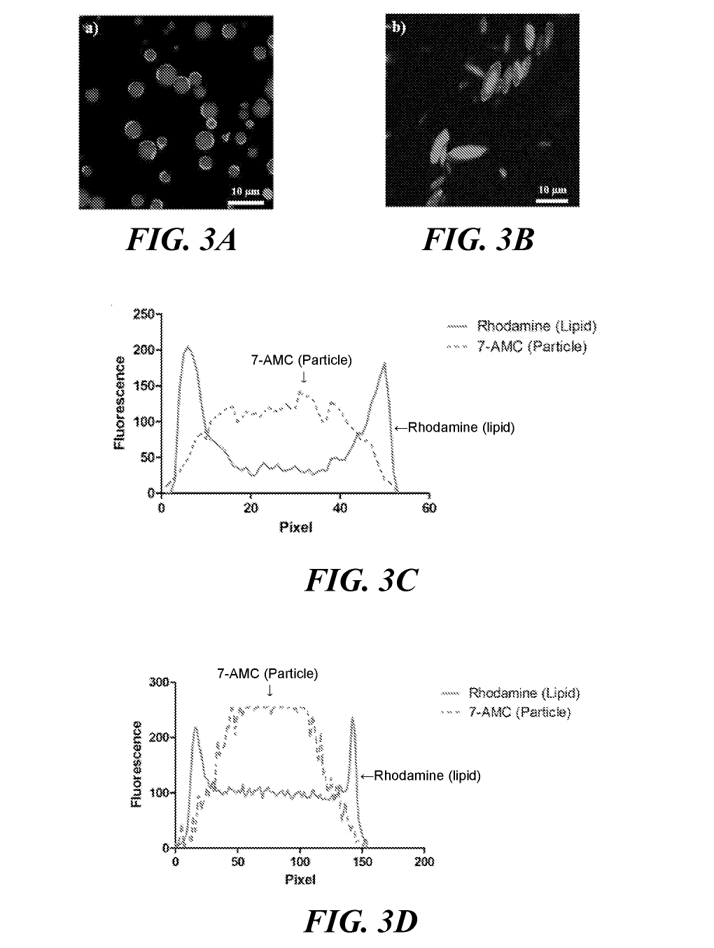

It was first desired to validate the capabilities to generate ellipsoidal and spherical SLBs utilizing poly (lactic-co-glycolic) acid for polymeric structural support. Spherical microparticles were generated by emulsion. The particles were sized by Image J analysis of SEM micrographs (FIG. 2A) and were determined to be 3.2 μm+/−1.2 μm (FIG. 2B). To confirm the maintenance of particle shape during the fabrication process, aspect ratio analysis of SEM micrographs of non-spherical particles coated with a lipid bilayer was performed (FIG. 2C). The aspect ratio of the ellipsoidal particles was measured to be 3.3+/−0.6, which was near the predicted value of 2.8 as computed previously for a two-fold stretched thin film (FIG. 2D and FIG. 2E; Sunshine et al., 2014). To confirm the presence of SLB, particles encapsulating 7-amino-4-methyl coumarin (7-AMC) were made to visualize the core of the lipid polymer hybrid particle. After thin-film stretching, the particles were coated with fluorescent liposomes containing rhodamine. Confocal imaging of a representative batch of particles revealed that the 7-AMC fluorescence was concentrated at the center of the particle for both spherical (FIG. 3A) and ellipsoidal (FIG. 3B) SLBs. The lipid-rhodamine signal, however, was localized to the exterior of the particle for both the ellipsoidal and spherical SLBs. In addition, it encircled the entire particle, indicating an even distribution of lipids across the particle surface. Profile analysis of each sample revealed that for both the spherical (FIG. 3C) and the ellipsoidal (FIG. 3D) that the maximum signal from the lipid-rhodamine conjugate was localized to the exterior of the particle utilizing the 7-AMC as a reference point.

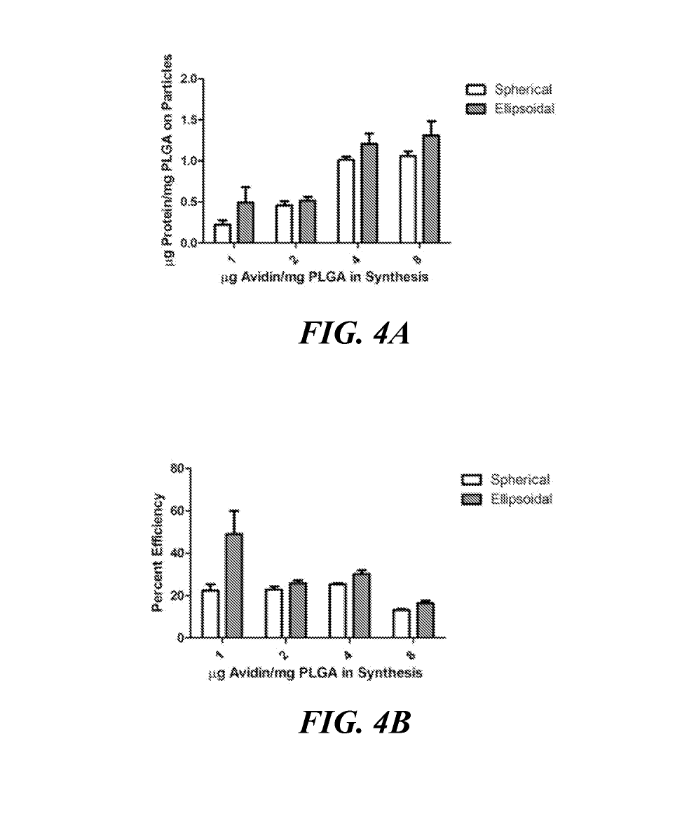

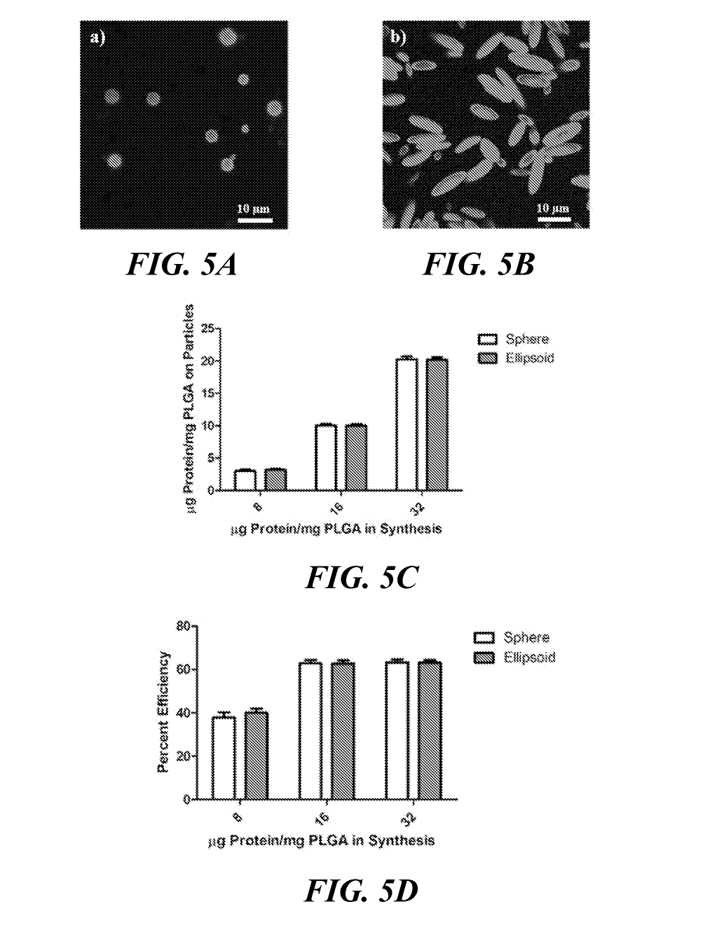

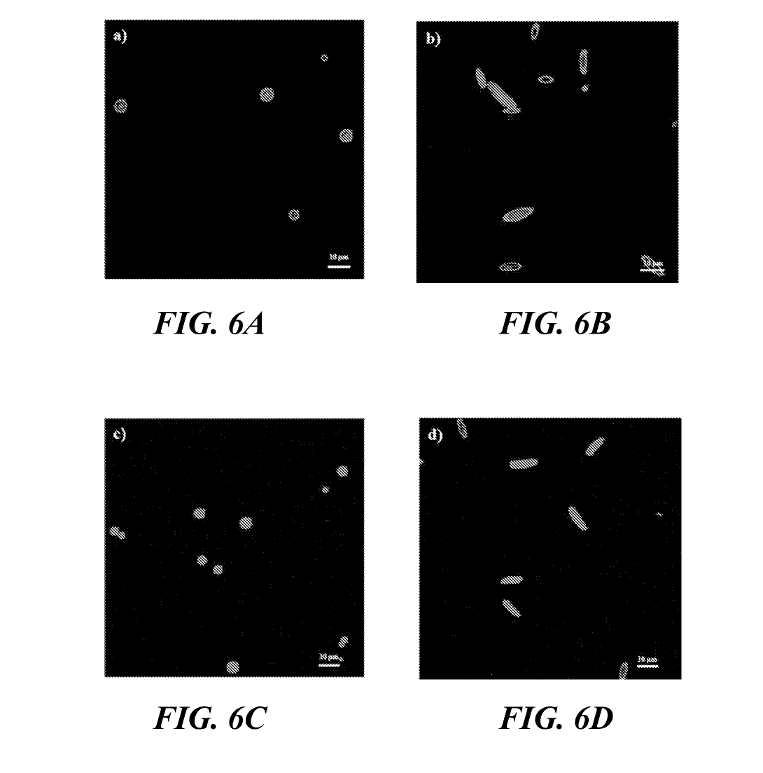

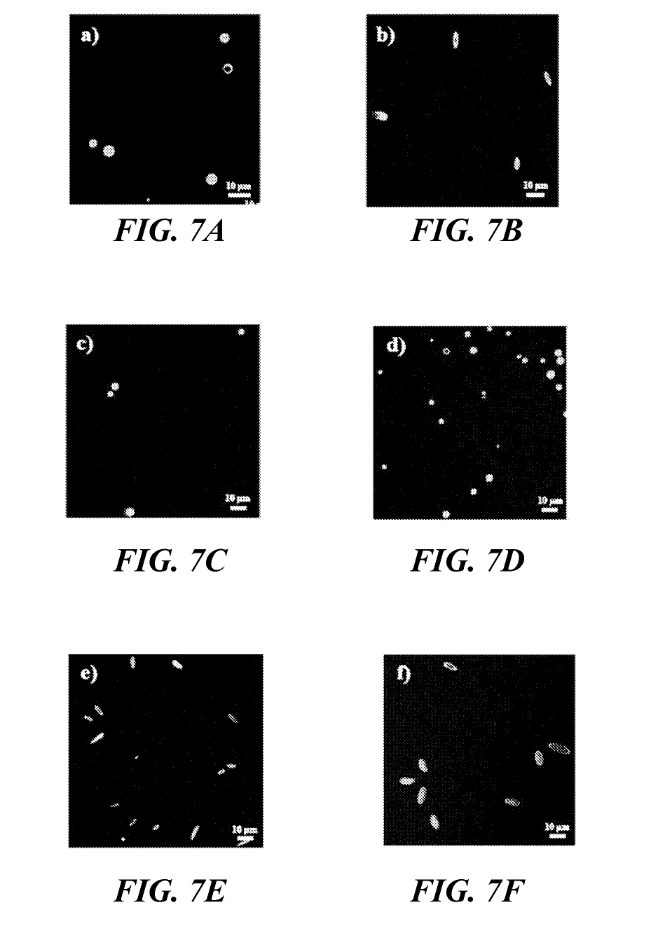

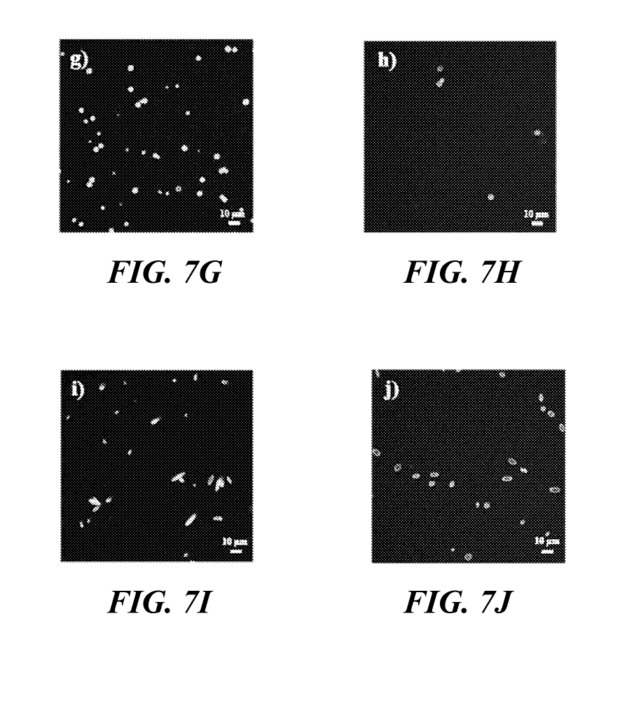

Upon successful validation of the SLB synthesis, the ability to conjugate target bioactive molecules to the surface of the supported lipid bilayers of both shapes was enabled. For many applications, such as cell type-specific targeting, this approach involves the conjugation of antibodies that recognize tumor-associated antigens (TAAs) to the SLB. In other cases, fluorescent moieties are conjugated to the particle to assist in the visualization and imaging of the particle or to study the fluidic properties of the SLB coating. To serve as an adapter for any target protein of small molecule, a thiolated avidin protein was conjugated to the surface of the SLB using maleimide-functionalized lipids added into the liposome during synthesis. Addition of this thiol avidin was confirmed by prereacting it with a biotinylated fluorescein conjugate and conjugating it to the surface (FIG. 4). SLBs functionalized with avidin can then be used to conjugate any biotinylated molecule. For an initial representative example, a biotinylated Cy5 molecule was used and imaged for both spherical (FIG. 5A) and ellipsoidal SLBs (FIG. 5B) encapsulating 7-AMC. As shown in the confocal micrographs of these particles, there was localization of the Cy5 signal to the exterior of the particle using 7-AMC as a reference point. Lyophilization stability of the SLB (FIG. 6) and time course stability of the SLBs (FIG. 7) were evaluated and determined to be stable in both situations.

Once capture of a biotinylated fluorophore was confirmed, a biotinylated antibody for murine CD28, an important co-stimulatory surface protein in the activation of lymphocytes, was evaluated. Through analysis of protein content in the supernatant of the wash steps, it was determined that the biotinylated antibody was captured on the surface of the particle and the conjugation procedure was dose dependent (FIG. 5C). In addition, the efficiency of conjugation was evaluated to be 50-70%, across the doses of protein in synthesis (FIG. 5D).

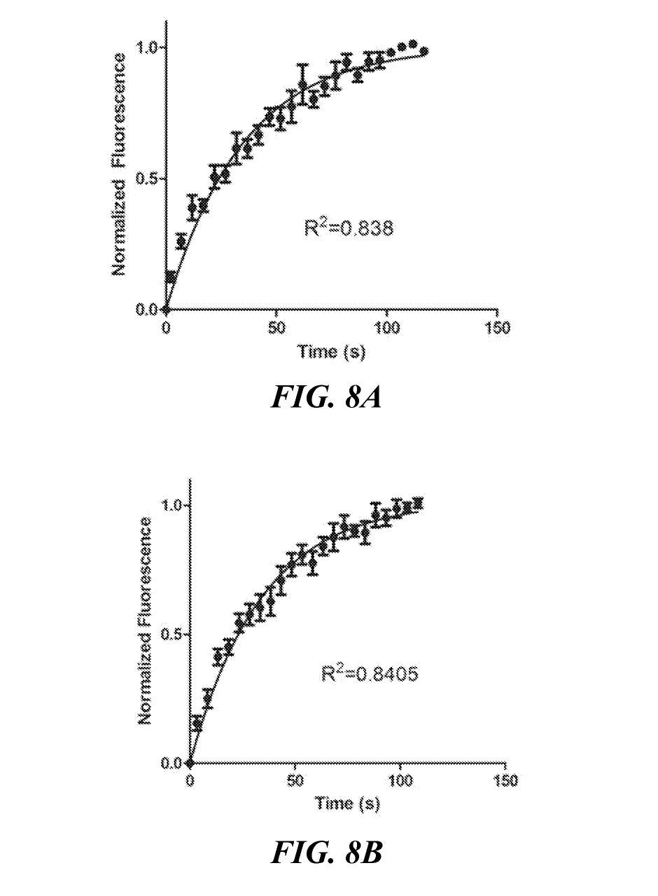

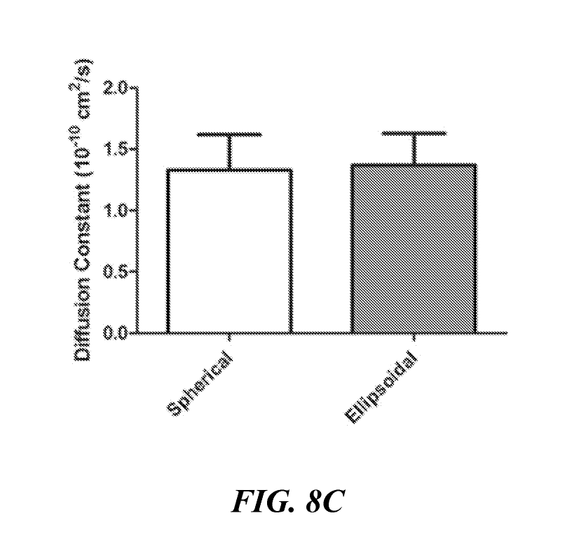

An important feature of the SLB is the capability to mimic natural membrane fluidity. One instance where this has been determined to be important is in the presentation of immunostimulatory ligands by artificial antigen presenting cells. Natural antigen presenting cells undergo dymanic rearrangement of their surface receptors upon engagement with a cognate T-Cell, and the lateral fluidity of these T-Cells has been deemed important for T-Cell activation (Meyer, Sunshine, and Green, 2015). To that end, the fluidity of the spherical and ellipsoidal SLB was evaluated by utilizing the fluorescence recovery after photobleaching technique (FRAP) as demonstrated previously (Bershteyn et al., 2008). Briefly, a particle was located under confocal microscopy. A small region of the lipid bilayer approximately 1 μm in diameter was identified and bleached using repeated cycles at maximum power of laser exposure. The resulting bleached region was monitored over time to determine the recovery of the fluorescent signal as a result of labeled lipids diffusing into the photobleached zone. Both spherical (FIG. 8A) and ellipsoidal (FIG. 8B) particles were noted to have membrane fluidity as evidenced by recovery of membrane fluorescence within 150 s by FRAP. The diffusion constant were evaluated by a normalization and fit of the signal recovery data (FIG. 8C), and diffusion constants were determined for both spherical and ellipsoidal SLBs. Both SLBs had statistically similar diffusion constants and the recovery time was on the same order of magnitude (10⁻¹⁰ cm²s⁻¹) of proteins on natural, biological membranes (Lenaz, 1987).

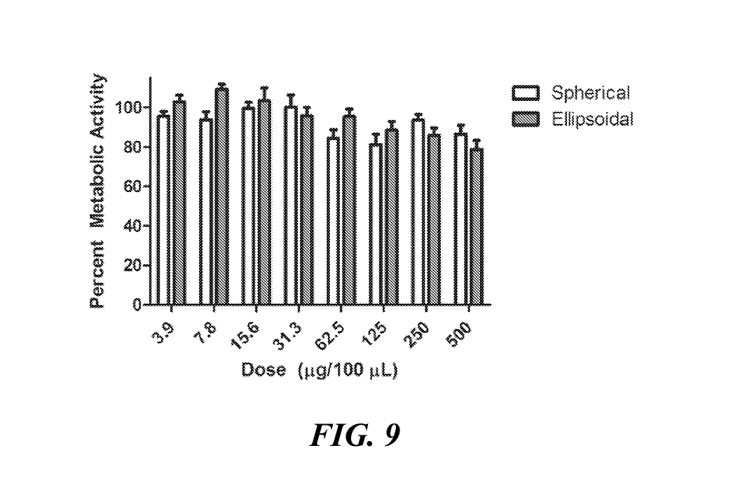

One advantage of the ellipsoidal particle over the spherical particle is the capability to evade non-specific uptake pathways. To verify these properties were maintained in the ellipsoidal SLBs, RAW 264.7 murine macrophages were used to mimic phagocytosis based elimination of particles in the reticuloendothelial system. The spherical and ellipsoidal SLB were compared in non-specific phagocytosis to determine if this new class of supported lipid bilayers could resist phagocytosis similar to what has been previously described (Sharma et al., 2010). Particles were formulated encapsulating a fluorescent dye and were coated with a lipid bilayer and conjugated to avidin via the thiol/maleimide chemistry. The SLBs were incubated with macrophages for 4 hours to permit for phagocytosis to occur. Viability of the macrophages was unaltered during this incubation, as evidenced by a cell titer assay of both the spherical and ellipsoidal lipid bilayers at the end of 4 hours (FIG. 9). At the conclusion of 4 hours, the macrophages were either fixed and stained for confocal imaging or removed from the plate by tituration for flow cytometry analysis.

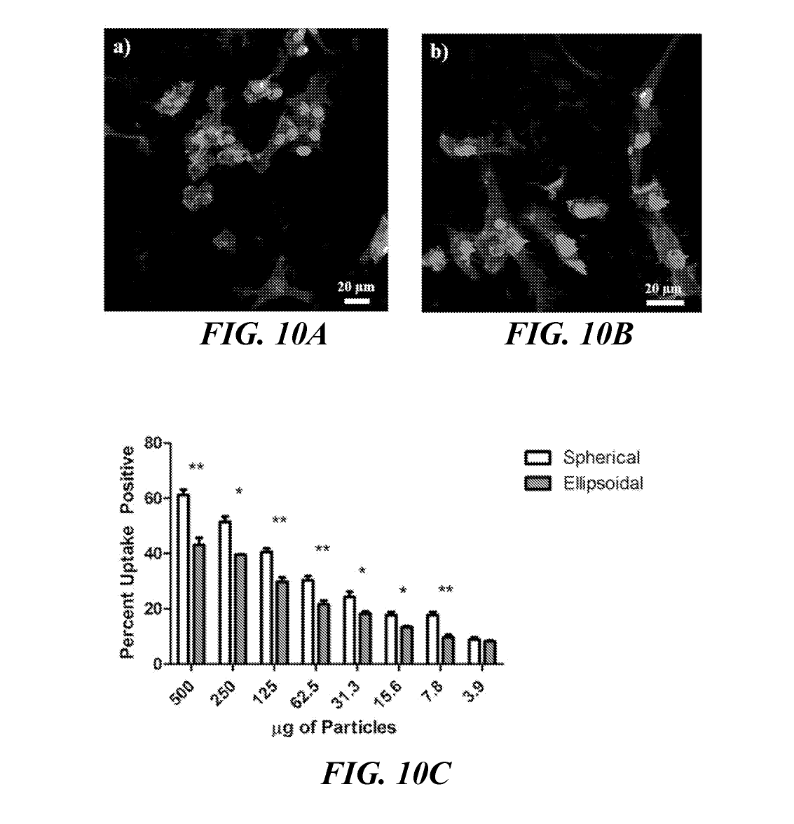

Confocal imaging analysis yielded a qualitative comparison of spherical vs ellipsoidal SLB phagocytosis (FIG. 10A vs. FIG. 10B). In all cases examined by microscopy, spherical SLBs were phagocytosed at a higher rate and in greater number compared to the ellipsoidal SLBs. Flow cytometry analysis of the cells after 4 hours of particle treatment supported these trends (FIG. 10C). As demonstrated with confocal imaging, both types of particles were capable of being internalized. However, there was a noted difference between the spherical and ellipsoidal samples. Across multiple doses of particles administered to the cells, there was a statistically significant (as determined by the one-tailed Student-T test) decrease in internalization rate of ellipsoidal SLBs compared to spherical SLBs. This is the same trend, which has previously been described in the literature (Lenaz, 1987) and confirms that the presently disclosed ellipsoidal SLBs maintain the advantageous biological properties of a non-spherical particle.

### Example 4

### Summary

In summary, a procedure to synthesize biomimetic ellipsoidal supported lipid bilayers has been successfully developed. Combining the previously developed procedures for thin-film stretching, and supported lipid bilayer synthesis, the presently disclosed subject matter has enabled the capability for surface presentation of biologically relevant proteins on a fluidic synthetic lipid membrane, supported by particles of different shapes. In addition, this platform has been verified to maintain the advantageous aspects of the non-spherical particle, specifically the capability to resist macrophage phagocytosis. This technology will allow for more accurate mimicry of natural cells through the presentation of laterally mobile proteins on the surface of anisotropic biodegradable particles. Examples of its use include creating synthetic particles that mimic biological cells, such as anisotropic supported lipid bilayer based artificial antigen presenting cells, and anisotropic supported lipid bilayer nanoparticles for cancer targeted drug delivery.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein

Ashley, C. E., et al., The targeted delivery of multicomponent cargos to cancer cells by nanoporous particle-supported lipid bilayers. Nature Materials 2011, 10 (389-397).

Bershteyn, A., et al., Polymer-supported lipid shells, onions, and flowers. Soft Matter 2008, 4 (1787-179).

Canelas, D. A., et al., Top-down particle fabrication: control of size and shape for diagnostic imaging and drug delivery. Wiley Interdisciplinary Reviews: Nanomedicine and Nanobiotechnology 2009, 1 (391-404).

Champion, J. A., et al., Particle shape: a new design parameter for micro-and nanoscale drug delivery carriers. J. Controlled Release 2007, 121(3-9).

Champion, J. A., et al., Making polymeric micro-and nanoparticles of complex shapes. Proc. Nat. Acad. Sci. 2007, 104 (11901-11904).

Cheow, W. S. and Hadinoto, K., Factors affecting drug encapsulation and stability of lipid-polymer hybrid nanoparticles. Colloids and Surfaces B: Biointerfaces 2011, 85 (214-220).

Chu, K. S., et al., Particle replication in nonwetting templates nanoparticles with tumor selective alkyl silyl ether docetaxel prodrug reduces toxicity. Nano Letters 2014, 14 (1472-1476).

Hadinoto, K., et al., Lipid-polymer hybrid nanoparticles as a new generation therapeutic delivery platform: a review. Eur. J. Pharm. Biopharm. 2013, 85 (427-443).

Hasan, W., et al., Delivery of multiple siRNAs using lipid-coated PLGA nanoparticles for treatment of prostate cancer. Nano Letters 2011, 12 (287-292).

Ho, C., et al., Preparation of monodisperse ellipsoidal polystyrene particles. Colloid and Polymer Science 1993, 271 (469-479).

Jiang, X., et al. String-like micellar nanoparticles formed by complexation of PEG-b-PPA and plasmid DNA and their transfection efficiency. Pharm. Res. 2011, 28 (1317-1327).

Jiang, X., et al., Plasmid-templated shape control of condensed DNA-block copolymer nanoparticles. Advanced Materials 2013, 25 (227-232).

Kang, M., et al., Simplified equation to extract diffusion coefficients from confocal FRAP data. Traffic 2012, 13(1589-1600).

Karagoz, B., et al., Polymerization-Induced Self-Assembly (PISA)-control over the morphology of nanoparticles for drug delivery applications. Polymer Chemistry 2014, 5 (350-355).

Lenaz, G., Lipid fluidity and membrane protein dynamics. Bioscience Reports 1987, 7 (823-837).

Meyer, J. J. Green, WIREs Nanomed. Nanobiotechnol. 2015, doi:10.1002/wnan.1348.

Meyer, R. A., R. S. Meyer, and J. J. Green, J. Biomed. Mater. Res. Part A 2015, 103, 2747-2757.

Meyer, R. A., J. C. Sunshine, J. J. Green, Trends Biotechnol. 2015, 33, 514-524.

Meyer, R. A., J. C. Sunshine, K. Perica, A. K. Kosmides, K. Aje, J. P. Schneck, J. J. Green, Small 2015, 11, 1519-1525. Sharma, G., et al., Polymer particle shape independently influences binding and internalization by macrophages. J. Controlled Release 2010, 147 (408-412).

Sunshine, J. C., et al., Particle shape dependence of CD8+ T cell activation by artificial antigen presenting cells. Biomaterials 2014, 35 (269-277).

Wang, H., et al. PLGA/polymeric liposome for targeted drug and gene co-delivery. Biomaterials 2010, 31 (8741-8748).

Willem, J., Engineering of lipid-coated PLGA nanoparticles with a tunable payload of diagnostically active nanocrystals for medical imaging. Chem. Comm. 2012, 48 (5835-5837).

Xu, J., et al., RNA replicon delivery via lipid-complexed PRINT protein particles. Mol. Pharm. 2013, 10 (3366-3374).

Although the foregoing subject matter has been described in some detail by way of illustration and example for purposes of clarity of understanding, it will be understood by those skilled in the art that certain changes and modifications can be practiced within the scope of the appended claims.

1. A non-spherical biomimetic artificial cell comprising:
(a) a three-dimensional microparticle or nanoparticle having an asymmetrical shape defined by a dimension (a) along an x-axis, a dimension (b) along a y-axis, and a dimension (c) along a z-axis, wherein at least one of (a), (b), or (c) is not equal to at least one other dimension (a), (b), or (c);
(b) a supported lipid bilayer (SLB); and
(c) at least one biomolecule conjugated to the SLB.

2. The non-spherical biomimetic artificial cell of claim 1 wherein the asymmetrical shape has at least one surface having a radius of curvature along at least one axis selected from one of the following ranges: (a) about 1 nm to about 10 nm; (b) about 11 nm to about 100 nm; (c) about 101 nm to about 400 nm; (d) about 401 nm to about 1 um; (e) about 10 μm to about 20 μm; (f) about 20 μm to about 100 μm; and (g) about 101 μm to about 1 mm.

3. (canceled)

4. The non-spherical biomimetic artificial cell of claim 1, wherein the artificial cell has an ellipsoidal shape.

5. The non-spherical biomimetic artificial cell of claim 4, wherein the three-dimensional microparticle or nanoparticle comprises an ellipsoid selected from the group consisting of:
a prolate ellipsoid, wherein the dimension (a) along the x-axis is greater than the dimension (b) along the y-axis, and wherein the dimension (b) along the y-axis is equal to the dimension (c) along the z-axis, such that the prolate ellipsoid can be described by the equation a>b=c;
a tri-axial ellipsoid, wherein the dimension (a) along the x-axis is greater than the dimension (b) along the y-axis, and wherein the dimension (b) along the y-axis is greater than the dimension (c) along the z-axis, such that the tri-axial ellipsoid can be described by the equation a>b>c; and
an oblate ellipsoid, wherein the dimension (a) along the x-axis is equal to the dimension (b) along the y-axis, and wherein the dimension (b) along the y-axis is greater than the dimension (c) along the z-axis, such that the oblate ellipsoid can be described by the equation a=b>c.

6. The non-spherical biomimetic artificial cell of claim 1, wherein the three-dimensional microparticle or nanoparticle comprises a material having one or more of the following characteristics:
(i) one or more degradable linkages;
(ii) a stretchable modulus; and
(iii) a glass transition temperature such that the material comprising the three-dimensional microparticle or nanoparticle is a solid at room temperature and/or body temperature.

7. The non-spherical biomimetic artificial cell of claim 6, wherein the degradable linkage is selected from the group consisting of an ester linkage, a disulfide linkage, an amide linkage, an anhydride linkage, and a linkage susceptible to enzymatic degradation.

8. The non-spherical biomimetic artificial cell of claim 1, wherein the microparticle or nanoparticle comprises a biodegradable polymer or blends of polymers selected from the group consisting of poly(lactic-co-glycolic acid) (PLGA), poly(beta-amino ester) (PBAE), polycaprolactone (PCL), polyglycolic acid (PGA), polylactic acid (PLA), poly(acrylic acid) (PAA), poly-3-hydroxybutyrate (P3HB), and poly(hydroxybutyrate-co-hydroxyvalerate).

9. The non-spherical biomimetic artificial cell of claim 8, wherein the microparticle or nanoparticle further comprises a biodegradable polymer or blends of polymers blended with a nondegradable polymer.

10. The non-spherical biomimetic artificial cell of claim 1, wherein the microparticle or nanoparticle has an aspect ratio ranging from about 1.1 to about 5.

11. The non-spherical biomimetic artificial cell of claim 1, wherein the at least one biomolecule conjugated to the SLB is found on the surface of the SLB.

12. The non-spherical biomimetic artificial cell of claim 1, wherein the at least one biomolecule conjugated to the SLB comprises at least one entity selected from the group consisting of a drug, therapeutic agent, protein, peptide, sugar and polysaccharide.

13. The non-spherical biomimetic artificial cell of claim 12, wherein the at least one biomolecule is a protein or a fragment thereof.

14. The non-spherical biomimetic artificial cell of claim 13, wherein the protein or a fragment thereof is a biotin-binding protein or fragment thereof.

15. The non-spherical biomimetic artificial cell of claim 14, wherein the biotin-binding protein or fragment thereof is selected from the group consisting of avidin, streptavidin, neutravidin, and fragment thereof.

16. The non-spherical biomimetic artificial cell of claim 1, wherein the SLB retains lateral diffusive properties as compared to the SLB of a similar spherical biomimetic artificial cell.

17. The non-spherical biomimetic artificial cell of claim 1, wherein the non-spherical biomimetic artificial cell has one or more of the following properties:
(a) a membrane fluidity selected from the group consisting of 10e-7 to 10e-8 cm²/s, 10e-8 to 10e-9 cm²/s, 10e-9 to 10e-10 cm²/s, 10e-10 to 10e-11 cm²/s, and 10e-11 to 10e-12 cm²/s;
(b) is more resistant to phagocytosis as compared to a similar spherical biomimetic artificial cell; and
(c) is capable of interacting with a T cell receptor.

18. (canceled)

19. (canceled)

20. A kit comprising a non-spherical biomimetic artificial cell of claim 1.

21. A method for administering a drug to a subject, the method comprising:
(a) providing a non-spherical biomimetic artificial cell comprising:
(i) a three-dimensional microparticle or nanoparticle having an asymmetrical shape defined by a dimension (a) along an x-axis, a dimension (b) along a y-axis, and a dimension (c) along a z-axis, wherein at least one of (a), (b), or (c) is not equal to at least one other dimension (a), (b), or (c); and
(ii) a supported lipid bilayer (SLB) functionalized with a biotin-binding protein or fragment thereof;
(b) providing a biotinylated drug;
(c) conjugating the biotinylated drug to the biotin-binding protein or fragment thereof on the non-spherical biomimetic artificial cell; and
(d) administering the biotinylated drug conjugated to the biotin-binding protein or fragment thereof on the non-spherical biomimetic artificial cell to the subject.

22. The method of claim 21, wherein the biotinylated drug is a biotinylated antibody.

23. The method of claim 22, wherein the biotinylated antibody is specific for CD28 or the T cell receptor (TCR).

24. The method of claim 21, wherein the subject is a human or a non-human animal.

25. (canceled)

26. The method of claim 21, wherein administering to the subject comprises administering one or more doses of the biotinylated drug conjugated to the non-spherical biomimetic artificial cell in an amount sufficient to treat a disease, disorder, or dysfunction.

27. The method of claim 21, wherein the administering is by oral ingestion, through injection, by infusion, through topical administration, through inhalation, through sublingual absorption, through rectal or vaginal delivery, subcutaneously, and combinations thereof.

28. A method for making non-spherical biomimetic artificial cells comprising a biodegradable three-dimensional microparticle or nanoparticle having an asymmetrical shape defined by a dimension (a) along an x-axis, a dimension (b) along a y-axis, and a dimension (c) along a z-axis, wherein at least one of (a), (b), or (c) is not equal to at least one other dimension (a), (b), or (c); a supported lipid bilayer (SLB); and at least one biomolecule conjugated to the SLB, the method comprising:
(a) providing or preparing a plurality of three-dimensional microparticles or nanoparticles having an asymmetrical shape;
(b) harvesting the plurality of three-dimensional microparticles or nanoparticles having an asymmetrical shape;
(c) providing a plurality of liposomes;
(d) fusing the plurality of liposomes to the plurality of three-dimensional microparticles or nanoparticles having an asymmetrical shape under sonication to form a plurality of three-dimensional microparticles or nanoparticles having an asymmetrical shape and a supported lipid bilayer (SLB); and
(e) conjugating a plurality of biomolecules to the surface of the plurality of three-dimensional microparticles or nanoparticles having an asymmetrical shape and a supported lipid bilayer (SLB) to form the non-spherical biomimetic artificial cells.

29. The method of claim 28, wherein the preparing a plurality of three-dimensional microparticles or nanoparticles having an asymmetrical shape comprises:
(a) providing or preparing a plurality of microparticles or nanoparticles;
(b) preparing a film comprising the plurality of microparticles or nanoparticles;
(c) stretching the film comprising the plurality of microparticles or nanoparticles to form a plurality of three-dimensional microparticles or nanoparticles having an asymmetrical shape; and
(d) harvesting the plurality of three-dimensional microparticles or nanoparticles having an asymmetrical shape.

30. The method of claim 29, wherein the film is heated before being stretched.

31. The method of claim 28, wherein the plurality of biomolecules is a plurality of biotin-binding proteins or fragments thereof, and the method further comprises adding a plurality of maleimide-functionalized lipids into the plurality of liposomes; thereby conjugating a thiolated biotin-binding protein or fragment thereof to the surface of the SLB.

32. The method of claim 28, wherein the biotin-binding protein or fragment thereof is selected from the group consisting of avidin, streptavidin, neutravidin, and fragment thereof.
